(19) United States
(12) Patent Application Publication
(10) Pub. No.: US 2024/0232787 A9
(43) Pub. Date: Jul. 11, 2024

(54) A PROCESS AND AN INFRASTRUCTURE FOR MONITORING A LOAD OF PERISHABLE PRODUCTS

(71) Applicant: Cryovac, LLC, Charlotte, NC (US)

(72) Inventors: Jvanohe Rizzi, Legnano (MI) (IT); Giorgia Francini, Arese (MI) (IT)

(21) Appl. No.: 18/278,122

(22) PCT Filed: Feb. 18, 2022
(86) PCT No.: PCT/IB2022/051471

§ 371 Date: Aug. 21, 2023

(30) Foreign Application Priority Data
Feb. 22, 2021 (IT) 102021000004043

(51) Int. Cl. G06Q 10/0832; G01K 13/00; G01N 33/02

(52) U.S. Cl. CPC G06Q 10/0832; G01K 13/00; G01N 33/02

(57) ABSTRACT

A process of monitoring a load of perishable products placed in a load volume includes a first level procedure of determining values of a first parameter over time based on measurements of a first parameter by first level sensors, determining if values of the first parameter comply or not with a predetermined first level acceptability criterion and, if the values of the first parameter comply with the predetermined first level acceptability criterion, repeating the execution of the first level procedure. The process also includes a second level procedure executed if the values of the first parameter do not comply with the predetermined first level acceptability criterion. The second level procedure involves determining which subgroups comply with a predetermined second level acceptability criterion by using at least some second level sensors in each respective subgroup of a plurality of subgroups of packages present in the load volume.

### FIELD OF THE INVENTION

The present invention refers to a process and an infrastructure for monitoring a load of perishable products. For example, the invention finds application in monitoring loads of perishable products such as foodstuffs from a production or sorting facility, where the products are packed and loaded onto a means of transport, during one or more transport stages to one or more destinations, where the products may be stored or displayed at a point of sale. The invention is particularly applicable to the monitoring of food products, fresh or frozen, but is generally applicable to any product likely to degrade over time: by way of example, pharmaceutical products, cosmetics, flowers, or industrial products that may degrade are included if, for example, subjected to inadequate thermal conditions.

### BACKGROUND ART

Various solutions are known that can monitor the storage conditions of products during their transport. For example, document WO2019/192741A1 concerns a monitoring system that uses ‘blockchain’ technology to certify that the quality of products remains adequate during transport. The system is designed to track products and detect product parameters that may have an impact on product quality. In particular, the sensors may include sensors attached directly to the product or its packaging and sensors positioned in the vehicle, which transmit measurement signals via radio to a monitoring control system. Document WO2019245717A1 concerns a monitoring system in which an indicator is attached to a perishable item or its packaging. The indicator includes ink marks that can be activated by a temperature change above a given threshold range. Activation causes an irreversible alteration of the temperature-sensitive ink marks providing a visible indication of previous temperature changes. In other words, once the indicator is activated and an aspect of the indicator alters to indicate the occurrence/detection of a temperature outside a threshold temperature range, the alteration is permanent and irreversible. A reader can then scan the indicators and inform a control network. A further known solution is described in U.S. Ser. No. 10/521,806B2, which describes a blockchain-based method for monitoring the product temperature during several stages of product life. The method involves the following steps:
requirements for a product are received and stored by a smart label (e.g. RFID) fixed to a package containing the product; the requirements can be generated and stored in a block of the blockchain by a product manufacturer;
the smart label also receives, from a temperature sensor, a temperature of the product (one or more temperature sensors can be placed inside and/or outside the package, and can be configured for transmitting temperature signals to the smart label);
the product temperature is compared with a temperature or temperature range specified for the product saved in the smart label memory;
the product temperature and the time the product temperature is received, are sent to the blockchain if the product temperature is outside a specified temperature range.

In practice, the smart label constantly receives the actual temperatures of the product from one or more sensors and compares the actual temperatures with the requirements. When the actual temperatures are greater or lower a specified temperature threshold, the actual temperatures and their corresponding recorded times can be transmitted to the blockchain.

In the known solutions described above, several types of sensors are therefore fixed to the product or its packaging and capable of sending information about the thermal condition of each product to a blockchain control system that supervises the status and therefore the quality of each product.

Although the solutions described are interesting, they are not free from drawbacks.

Indeed, the sensors typically used have relatively high costs and therefore the use of such technologies is not often suitable for use in all industries. In particular, in the food industry, packaged products have a relatively low cost that rarely justifies the use of technologies such as those described.

Moreover, managing the large amount of information from the sensors is often complex due to the large amount of data that is transmitted during monitoring and therefore must be sent and managed by a remote system.

Finally, specifically in the field of distributed registers, there is the problem of ensuring that the data coming from the sensors are true and accurate.

### OBJECT OF THE INVENTION

Object of the present invention is therefore to solve at least one of the drawbacks and/or limitations of the previous solutions.

A first object of the invention is to provide a process and an infrastructure for monitoring a load of perishable products, which can be implemented at relatively low cost and therefore has a negligible impact on the cost of the individual product.

It is also object of the present invention to provide a process and an infrastructure for monitoring one or more loads of perishable products by reducing the number of measurements and thereby reducing the amount of data to be transmitted and monitored.

It is object of the present invention to provide a process and an infrastructure for monitoring loads of perishable products which guarantees a high standard of reliability, so as to certify the quality of the monitored products in an indisputable manner.

It is a further object of the present invention to provide a process and an infrastructure for monitoring loads of perishable products which is well suited for monitoring the cold chain of food products, whether frozen or fresh. Finally, it is an object of the invention to provide a process and an infrastructure for monitoring loads of perishable products which can be installed relatively easily and which requires a small number of maintenance interventions.

These objects and others, which will become more apparent from the following description, are substantially achieved by a process and an infrastructure for monitoring a load of perishable products according to one or more of the following claims and/or aspects.

### SUMMARY

Aspects of the invention are described below.

A first aspect concerns a process of monitoring a load of perishable products, said load comprising a multiplicity of products housed in respective packages (2) and placed in a load volume of a storage unit or a conveyor, wherein:
the load volume (3) is provided with a predetermined number of first-level sensors (4) configured for detecting a first parameter relative to an environment present in the load volume (3) itself,
the load volume (3) comprises a plurality of subgroups (13) of said multiplicity of products present in that load volume, wherein each subgroup (13) comprises a plurality of products which are, in number, a fraction, and in particular a submultiple of said multiplicity of products present in the load volume (3),
each of the packages (2) or of the products housed in said packages (2) is associated with at least one second level sensor (5), each second level sensor (5) being sensitive to at least one alteration of a second parameter relative to each package (2) or to an environment inside each package (2).

In a 2nd aspect according to the 1st aspect, said process includes the execution of a first level procedure comprising the following steps:
determining values assumed by said first parameter over time based on detections of said first parameter carried out by said predetermined number of first level sensors (4),
establishing if said values of the first parameter comply with or do not comply with a predetermined first level acceptability criterion,
if the values of the first parameter comply with the predetermined first-level acceptability criterion, repeating the execution of the first level procedure.

In a 3rd aspect according to the preceding aspect said process includes a second level procedure which is performed if the values of the first parameter do not comply with the predetermined first level acceptability criterion, the second level procedure comprising the following steps:
establishing which subgroups comply with a predetermined second level acceptability criterion, using at least some of the second level sensors (5) associated with the products or packages (2) in each respective subgroup (13).

In a 4th aspect according to the preceding aspect, the second level sensors (5) are of type different from that of the first level sensors (4).

In a 5th aspect according to any one of the preceding aspects the load volume (3) comprises a plurality of subgroups (13) of products, each subgroup (13) being identifiable and distinct from the other subgroups, in particular wherein each subgroup is housed in a respective enclosure or placed on a respective pallet.

In a 6th aspect according to any one of the preceding aspects from the 2nd to 5th the process comprises the further step of certifying that all the products in the load volume (3) comply with a predetermined quality standard if, in a reference time interval during which the first level procedure is performed a plurality of times, the following is verified:
the values of the first parameter comply with the predetermined first level acceptability criterion during each execution of the first level procedure.

In a 7th aspect according to any one of the preceding aspects from the 3rd to 6th the process comprises the further step of certifying that the products in the load volume (3) all meet a predetermined quality standard if, in a reference time interval during which the first level procedure is performed a plurality of times, the following is verified:
the second level procedure is never performed.

In an 8th aspect according to any one of the preceding aspects from the 3rd to 7th the process comprises the further step of certifying that all the products in the load volume (3) comply with a predetermined quality standard if, in a reference time interval during which the first level procedure is performed a plurality of times, the following occurs:
if the second level procedure is performed, it is verified that all subgroups comply with the predetermined second level acceptability criterion.

In a 9th aspect according to any one of the previous three aspects, the reference time interval is equal to the duration of a transport of a load from a delivery point to a destination.

In a 10th aspect according to any one of the previous aspects from the 3rd to 9th the second level procedure includes the additional step of reporting subgroups that do not comply with the predetermined second level acceptability criterion.

In an 11th aspect according to any one of the preceding aspects from the 2nd to 10th, each of the first level sensors (4) comprises a respective control unit (9), a memory (10) and a transmitter (11), communicatively connected to each other in a direct or indirect manner.

In a 12th aspect according to the preceding aspect each of the first level sensors is configured for:
storing one or more values assumed over time by the first parameter and
transmitting such one or more values to a control supervisor (6) of the process configured for performing the first level procedure.

In a 13th aspect according to any one of the preceding aspects from the 2nd to 12th, each of the first level sensors (4) comprises a respective control unit (9), a memory and a transmitter, communicatively connected to each other in a direct or indirect manner.

In a 14th aspect according to any of the previous aspects from the 2nd to 13th, each of the first level sensors is configured for:
storing one or more values assumed over time by the first parameter and
performing the first level procedure,
transmitting to a control supervisor (6) of the process the values assumed over time by the first parameter if the values of the first parameter do not comply with the predetermined first level acceptability criterion.

In a 15th aspect according to any one of the preceding aspects from the 2nd to 14th the (or a) control supervisor is communicatively connected with the first level sensors (4) and it is housed in the load volume (3) or in a remote position with respect to the load volume (3).

In a 16th aspect according to any one of the preceding aspects from the 2nd to 15th each of said first level sensors comprises a Bluetooth transmitter, a WiFi transmitter, an RFID transponder tag, an NFC device.

In a 17th aspect according to any one of the preceding aspects from 2 to 16 the first parameter is the temperature or the temperature variation.

In an 18th aspect according to any one of the preceding aspects from the 2nd to 16th the first parameter is a parameter that is a function of the temperature, or of the temperature variation: e.g. humidity, conductivity or other air parameters may be used.

In a 19th aspect according to any one of the preceding aspects from the 2nd to 16th the first parameter is the concentration of a predetermined gas or mixture of gases at the position inside the load volume at which each first level sensor operates (4).

In a 20th aspect according to any one of the preceding aspects from the 2nd to 16th the first parameter is the concentration variation of a predetermined gas or gas mixture.

In a 21st aspect according to any one of the preceding aspects from the 2nd to 20th the first parameter is detected at the position inside the load volume in which each first level sensor (4) operates.

In a 22nd aspect according to the 17th or 18th aspect, the predetermined first level acceptability criterion is defined by a range of temperatures, or by a range of parameter acceptability values as a function of temperature or temperature variation.

In a 23rd aspect according to the 19th or 20th aspect the predetermined first level acceptability criterion is defined by a concentration range or concentration variation of a prefixed gas or gas mixture.

In a 24th aspect according to any one of the preceding aspects from the 2nd to 23rd a plurality of first level sensors (4) are distributed, optionally uniformly distributed, in the load volume.

In a 25th aspect according to any of the previous aspects from the 2nd to 24th, each of the first sensors (4) is associated with a unique identification code.

In a 26th aspect according to the preceding aspect, the process comprises the step of transmitting to a, or to the, control supervisor (6) of the process the values assumed over time by the first parameter associating the respective unique identification code of each of the first sensors to enable the control supervisor (6) to identify the values assumed over time by the first parameter detected by each of said first sensors (4).

In a 27th aspect according to any of the preceding aspects from the 3rd to 26th the second parameter is the temperature, or a temperature-dependent parameter, of each package (2) or of an environment inside each package.

In a 28th aspect according to any one of the preceding aspects from the 3rd to 26th the second parameter is the temperature of an outer surface of the package (2).

In a 29th aspect according to any one of the preceding aspects from the 3rd to 26th the second parameter is the temperature inside each package (2).

In a 30th aspect according to any one of the preceding aspects from the 3rd to 26th the second parameter is the concentration or concentration variation of a predetermined gas inside the package (2).

In a 31st aspect according to any one of the preceding aspects from the 3rd to 26th the second parameter is the concentration or concentration variation of a predetermined substance inside the package.

In 32nd aspect according to any one of the preceding aspects from the 3rd to 26th the second parameter is the degree of acidity present inside the package (2) or the variation of the degree of acidity in each package (2).

In a 33rd aspect according to the 27th or 29th aspect each of the second level sensors (5) associated with a respective package (2) comprises:
a component sensitive to temperature or to a parameter proportional to temperature arranged inside the package.

In a 34th aspect according to the 28th aspect each of the second level sensors (5) associated with a respective package (2) comprises:
component sensitive to temperature or to a parameter proportional to temperature carried by the product package, in particular the outer surface of the package.

In a 35th aspect according to the 30th aspect each of the second level sensors (5) associated with a respective package (2) comprises:
a component, sensitive to the concentration or concentration variation of a predetermined gas, arranged inside the package.

In a 36th aspect according to the 31st aspect each of the second level sensors (5) associated with a respective package (2) comprises:
a component, sensitive to the concentration of a given substance or to the variation of a given substance, arranged inside the package.

In a 37th aspect according to the 30th aspect each of the second level sensors (5) associated with a respective package (2) comprises:
a component, sensitive to the concentration or concentration variation of a predetermined gas, carried by the package,
In a 38th aspect according to the 31st aspect each of the second level sensors (5) associated with a respective package (2) comprises:
a component, sensitive to the concentration of a predetermined substance or to the variation of a predetermined substance, carried on the package.

In a 39th aspect according to the 32nd aspect each of the second level sensors (5) associated with a respective package (2) comprises:
a component sensitive to the pH or pH variation of the environment inside the package.

In a 40th aspect according to any one of aspects from the 3rd to 39th each of the second level sensors (5) associated with a respective package (2) is a disposable component applied to the package or arranged inside the package.

In a 41st aspect according to any one of aspects from the 3rd to 39th each of the second level sensors (5) associated with a respective package (2) is a component printed directly on the package, optionally on a plastic film or on a plastic or paper tray forming part of the package.

In a 42nd aspect according to any one of aspects from the 3rd to 41st each of the second level sensors (5) is configured for providing an indication, optionally visual, of yes/no type of the fact that the product inside the respective package has exceeded or not a certain temperature for a specific time interval.

In a 43rd aspect according to any one of aspects from the 3rd to 41st each of the second level sensors (5) is configured for providing an indication, optionally visual, proportional to the maximum temperature reached by the product inside the respective package.

In a 44th aspect according to any one of aspects from the 3rd to 41st each of the second level sensors (5) is configured for providing an indication, optionally visual, of yes/no type of the fact that the area of the package where the second level sensor is present has exceeded or not a certain temperature for a specific time interval.

In a 45th aspect according to any of aspects from the 3rd to 41st each of the second level sensors (5) is configured for providing an indication, optionally visual, proportional to the maximum temperature reached zone of the package where the second level sensor is present.

In a 46th aspect according to any one of aspects from the 3rd to 41st each of the second level sensors (5) is configured for providing an indication, optionally visual, of yes/no type of the fact that a certain concentration or concentration variation of a predetermined gas or gas mixture is exceeded or not within the respective package, optionally for a specific time interval.

In a 47th aspect according to any one of aspects from the 3rd to 41st each of the second level sensors (5) is configured for providing an indication, optionally visual, proportional to the maximum concentration of a predetermined gas or gas mixture reached inside the respective package.

In a 48th aspect according to any one of aspects from the 3rd to 41st each of the second level sensors (5) is configured for providing an indication, optionally visual, of yes/no type of the fact that a certain concentration or concentration variation of a predetermined substance or group of substances is exceeded or not within the respective package, optionally for a specific time interval.

In a 49th aspect according to any one of aspects from the 3rd to 41st each of the second level sensors (5) is configured for providing an indication, optionally visual, proportional to the maximum concentration of a predetermined substance or group of substances inside the respective package.

In a 50th aspect according to any one of aspects from the 3rd to 41st each of the second level sensors (5) is configured for providing an indication, optionally visual, of yes/no type of the fact that at a surface of the respective package a certain concentration or concentration variation of a predetermined gas or gas mixture is exceeded or not, optionally for a specific time interval.

In a 51st aspect according to any one of aspects from the 3rd to 41st each of the second level sensors (5) is configured for providing an indication, optionally visual, proportional to the maximum concentration of a predetermined gas or gas mixture reached at a surface of the respective package.

In a 52nd aspect according to any one of aspects from the 3rd to 41st each of the second level sensors (5) is configured for providing an indication, optionally visual, of yes/no type of the fact that a certain pH or pH variation has been exceeded or not inside the respective package, optionally for a specific time interval.

In a 53rd aspect according to any one of aspects from the 3rd to 41st each of the second level sensors (5) is configured for providing an indication, optionally visual, proportional to the maximum or minimum pH reached inside the respective package.

In a 54th aspect according to any one of aspects from the 3rd to 53rd each of the second level sensors (5) is configured for providing that the predetermined second level acceptability criterion is considered to be complied with for a given subgroup (13) only if the indication, optionally visual, provided by each of the second level sensors (5) used in the second level procedure for that subgroup complies with a respective verification test.

In a 55th aspect according to any one of aspects from the 3rd to 54th, the step of the second level procedure of determining which subgroups comply with a predetermined second level acceptability criterion includes:
identifying in each subgroup (13) a predetermined number of sample packs (12), said sample packs (12) being smaller in number than the total number of packs (2) in each subgroup (13), in particular where said sample packs (12) are less than half the number of packs (2) in each subgroup (13),
establishing that a subgroup fulfils the predetermined second level acceptability criterion exclusively by using the second level sensors (5) associated with the sample packages (12), or the products contained in the sample packages (12), present in the respective subgroup (13).

In a 56th aspect according to the preceding aspect the sample packages (12) of each subgroup (13) are placed in an outer peripheral area of the subgroup.

In a 57th aspect according to any one of the two preceding aspects, each subgroup has packages which are placed side by side and superimposed on each other to form an ideal cube or an ideal parallelogram, and in which each of the sample packages (12) is placed at a respective edge or vertex of the ideal cube or parallelogram.

In a 58th aspect according to the preceding aspect the sample packages (12) are two or more in number, optionally from 3 to 6, for each subgroup (13).

In a 59th aspect according to any one of aspects from the 3rd to 58th each package (2), in particular each of the second level sensors (5) in each package, is associated with a unique identification code.

In a 60th aspect according to any one of aspects from the 3rd to 59th the step of determining which subgroups comply with a predetermined second level acceptability criterion includes, for each subgroup:
querying or observing one or more second level sensor(s) (5) of the packages (2) of subgroup (13), and
sending to one, or the, control supervisor (6) of the process at least one indication, related to the second parameter, emitted or displayed by each of the queried or observed second level sensors (5) by associating to it the unique identification code of the respective package whose second sensors have been queried or observed.

In a 61st aspect according to any one of the two preceding aspects, the step of identifying a predetermined number of sample packages in each subgroup (12) involves using a query protocol comprising a list of unique packages identification codes and selecting those from the list in the query protocol as sample packs.

In a 62nd aspect according to any one of the three preceding aspects, the second level procedure comprises a step of verifying that the sample packages (12) identified in each subgroup (13) are all and only those present in a list of the query protocol including the unique identification codes of the sample packages (12) to be selected.

In a 63rd aspect according to the preceding aspect the verification phase is carried out by a control supervisor (6).

In a 64th aspect according to any one of the preceding two aspects, the query protocol is stored in the control supervisor (6).

In a 65th aspect according to any one of aspects from the 3rd to 64th the indication provided by each of the second level sensors (5) is read by at least one detector (8) communicatively connected to the control supervisor (6) and is transmitted to the latter.

In a 66th aspect according to the preceding aspect the detector (8) comprises:
at least an optical scanner or camera if the indication is of visual type, or
at least one radio wave detector if the indication is of electromagnetic type, or
at least one acoustic detector, if the indication is of sound type.

In a 67th aspect according to any one of aspects from the 3rd to 66th wherein the/a control supervisor (6) comprises one or more digital electronic processor and/or one or more analogue electronic processors and wherein the process steps according to one or more of the preceding aspects are performed by, or under the control of, the control supervisor (6).

In a 68th aspect according to any one of aspects from the 3rd to 67th the unique identification code relating to each first level sensor (4) is communicated, for example periodically or at regular intervals or following a command, to a distributed registry (16) of blockchain type connected to said control supervisor (6).

In a 69th aspect according to any one of aspects from the 3rd to 68th the pair of values, given by the first parameter value and the sampling instant, detected by each first level sensor (4) during the execution of the first level procedure, is communicated, for example periodically or at regular intervals or following a command, to a distributed register (16) of blockchain type connected to said control supervisor (6).

In a 70th aspect according to any one of aspects from the 3rd to 69th the unique identification code of each second level sensor (5) is communicated, e.g. periodically or at regular intervals or following a command, to a distributed registry (16) of blockchain type connected to said control supervisor (6).

In a 71st aspect according to any one of aspects from the 3rd to 70th the pair of values, given by indication related to the second parameter and instant of detection, recorded by the detector (8) for each queried second level sensor is communicated, for example periodically or at regular intervals or following a command, to a distributed register (16) of blockchain type connected to said control supervisor (6).

In a 72nd aspect according to any one of aspects from the 3rd to 71st the process comprises the further step of certifying that one or more subgroups (13) present in the load volume consist exclusively of products complying with a predetermined quality standard if, in a reference time interval during which the first level procedure is performed a plurality of times, the following occurs:
the values of the first parameter comply with the predetermined first level acceptability criterion each time the first level procedure is performed.

In a 73rd aspect according to any one of aspects from the 3rd to 71st the process comprises the further step of certifying that one or more subgroups (13) present in the load volume consist exclusively of products complying with a predetermined quality standard if, in a reference time interval during which the first level procedure is performed a plurality of times, the following occurs:
the values of the first parameter do not comply with the predetermined first level acceptability criterion during at least one execution of the first level procedure, and
when the second level procedure is performed, it is verified that said one or more subgroups (13) comply with the predetermined second level acceptability criterion.

In a 74th aspect according to one of the two preceding aspects, the reference time interval is equal to the duration of a transport of the load from a delivery point to a destination.

In a 75th aspect according to any one of aspects from the 3rd to 74th each of the second level sensors (5) associated with a respective package is
a disposable component attached to or inserted into the package.

In a 76th aspect according to any one of aspects from the 3rd to 74th each of the second level sensors (5) associated with a respective package is
a component printed directly on the package, optionally on a plastic film or on a plastic or paper tray forming part of the package (2).

In a 77th aspect according to any one of aspects from the 1st to 76th the load volume (3) is defined within a loading compartment of a land vehicle, ship, or aircraft, and where the monitoring process is performed continuously throughout the entire load transport from a loading delivery point to its destination.

In a 78th aspect according to any one of aspects from the 1st to 77th the products are fresh or frozen foodstuffs; or the products are pharmaceutical products, in particular pharmaceutical products comprising antiviral or antibacterial active ingredients which must be stored at temperatures at least below 0 CO.

In a 79th aspect according to any one of aspects from the 1st to 78th the package is a primary package directly housing the product, or wherein the package is a secondary package housing in turn a plurality of primary packages housing the product.

In an 80th aspect according to any one of aspects from the 1st to 79th the package, or at least the primary package, is a plastic film bag, or a plastic film envelope, or a tray with a plastic film wrapping on top, or a tray with a plastic film wrapping the product and the tray.

An 81st aspect concerns an infrastructure for monitoring a load of perishable products, said load comprising a multiplicity of products housed in respective packages (2) and placed in a load volume (3) of a storage unit or conveyor, wherein:
the load volume (3) is provided with a predetermined number of first level sensors (4) configured for detecting a first parameter relative to an environment present in the load volume itself,
the load volume (3) comprises a plurality of subgroups (13) of that plurality of products present in the load volume, wherein each subgroup in turn comprises a plurality of products which are, in number, a fraction and in particular a submultiple of that plurality of products present in the load volume,
each of the packages (2) or the products housed in said packages is associated with at least one second level sensor (5), each second level sensor (5) being sensitive to at least one alteration of a second parameter relative to an environment adjacent to or inside each package.

In an 82nd aspect according to the preceding aspect said infrastructure (1) comprises at least one control supervisor (6) configured for executing a first level procedure comprising the following steps:
receiving, or determining based on signals emitted by the first-level sensors, values assumed by said first parameter over time based on detections of the first parameter carried out by said predetermined number of first-level sensors (4),
establishing if said values of the first parameter comply with or do not comply with a predetermined first level acceptability criterion,
if the values of the first parameter comply with the predetermined first level acceptability criterion, repeating the execution of the first-level procedure.

The control supervisor (6) is housed in the load volume (3) or in a position remote from the load volume or it may comprise parts in the load volume and parts at a place remote from the load volume.

In an 83rd aspect according to the previous aspect said infrastructure is also configured for executing a second level procedure if the values of the first parameter do not comply with the predetermined first level acceptability criterion, the second level procedure comprising the following steps:
establishing which subgroups (13) comply with a predetermined second level acceptability criterion using indications relative to alteration of the second parameter detected by at least one part of the second level sensors (5) associated with the products or packages (2) present in each respective subgroup.

In an 84th aspect according to the preceding aspect the control supervisor (6) is configured for executing the second level procedure.

In an 85th aspect according to any of the preceding three aspects the control supervisor (6) is configured for:
being communicatively connected with the first level sensors (4) or with intermediate transmission units (7) communicatively interposed between the control supervisor (6) and the first level sensors (4), and
receiving the signals emitted by the first level sensors (4) or directly the values assumed by the first parameter over time.

In an 86th aspect according to any one of the aspects from the 81st to 85th the second level sensors (5) are of a different type from the first level sensors (4).

In an 87th aspect according to any one of the aspects from the 82nd to 86th the control supervisor (6) is configured for:
being communicatively connected with the second level sensors (5) or with a detector (8) configured for reading the indications emitted by the second level sensors (5) communicatively interposed between the control supervisor (6) and the second level sensors (5) and
receiving from said detector (8) or directly from said second level sensors (5) the indications relative to the alteration of the second parameter.

In an 88th aspect according to any one of aspects from the 82nd to 87th the control supervisor (6) is configured for executing the further step of certifying that the products in the load volume (3) all comply with a predetermined quality standard if, in a reference time interval during which the first level procedure is performed a plurality of times, the following is verified:
the values of the first parameter comply with the predetermined first level acceptability criterion during each execution of the first level procedure.

In an 89th aspect according to any one of aspects from the 82nd to 88th the control supervisor (6) is configured for executing the further step of certifying that the products in the load volume (3) all comply with a predetermined quality standard if, in a reference time interval during which the first level procedure is performed a plurality of times, the following occurs:
the second level procedure is never executed.

In a 90th aspect according to any one of aspects from the 82nd to 89th the control supervisor (6) is configured for executing the further step of certifying that the products in the load volume (3) all comply with a predetermined quality standard if, in a reference time interval during which the first level procedure is performed a plurality of times, the following occurs:
if the second level procedure is executed, it is verified that all subgroups comply with the predetermined second level acceptability criterion.

In a 91st aspect according to any one of the aspects from the 88th to 90th the reference time interval is equal to the duration of one transport of load from a delivery point to a destination.

In a 92nd aspect according to any one of the aspects from the 82nd to 91st the second level procedure includes the additional step of reporting subgroups that do not comply with the predetermined second level acceptability criterion.

In a 93rd aspect according to any one of the aspects from the 81st to 92nd the infrastructure includes the first level sensors.

In a 94th aspect according to any one of the aspects from the 81st to 93rd each of the first level sensors (4) comprises a respective control unit (9), a memory (10) and a transmitter (11), communicatively connected to each other directly or indirectly.

In a 95th aspect according to any one of the aspects from the 81st to 94th each of the first level sensors (4) is configured for:
storing one or more values assumed over time by the first parameter and
transmitting such one or more values to the control supervisor configured for performing the first level procedure.

In a 96th aspect according to any one of the aspects from the 81st to 95th each of the first level sensors (4) comprises a Bluetooth transmitter, a WiFi transmitter, an RFID transponder tag, an NFC device.

In a 97th aspect according to any one of the aspects from the 81st to 96th the first parameter is temperature, or a parameter which is a function of temperature, or the concentration of a predetermined gas or mixture of gases, or the concentration variation of a predetermined gas or mixture of gases.

In a 98th aspect according to any one of the aspects from the 81st to 97th said first parameter is detected at the position in the load volume wherein each first level sensor (4) operates.

In a 99th aspect according to any one of the aspects from the 82nd to the 98th said first parameter is the temperature and the predetermined first level acceptability criterion is defined by a temperature range.

In a 100th aspect according to any one of the aspects from the 82nd to 98th said first parameter is a parameter function of the temperature and said prefixed acceptability criterion is defined by a range of values of the parameter function of the temperature.

In a 101st aspect according to any one of the aspects from the 82nd to 98th said first parameter is the concentration of a prefixed gas or mixture of gases and said prefixed acceptability criterion is defined by a concentration range of a prefixed gas or mixture of gases.

In a 102nd aspect according to any one of the aspects from the 82nd to 98th said first parameter is the temperature variation and said prefixed acceptability criterion is defined by a range of values of the temperature variation.

In a 103rd aspect according to any one of the aspects from the 82nd to 98th said first parameter is the variation of concentration of a predetermined gas or mixture of gases and said predetermined acceptability criterion is defined by a range in the variation of concentration of a predetermined gas or mixture of gases.

In a 104th aspect according to any one of the aspects from the 82nd to 103rd the infrastructure comprises a plurality of first level sensors (4) distributed, optionally uniformly distributed, in the load volume (3), each of the first level sensors (4) being associated with a unique identification code.

In a 105th aspect according to the preceding aspect, the first level sensors (4) are configured for transmitting to the control supervisor (6) the values assumed over time by the first parameter associating the respective unique identification code of each of said first sensors, and wherein the control supervisor (6) is configured for identifying the values assumed over time by the first parameter detected by each of said first sensors.

In a 106th aspect according to any one of the aspects from the 81st to 105th wherein the second parameter is the temperature of each package (2) (for example of a surface thereof) or of an environment inside each package.

In a 107th aspect according to any one of the aspects from the 81st to 105th wherein the second parameter is a parameter that is a function of the temperature of each package (2) (for example of a surface thereof) or of an environment inside each package.

In a 108th aspect according to any one of the aspects from the 81st to 107th wherein the second parameter is the temperature of an external surface of the package (2).

In a 109th aspect according to any one of the aspects from the 81st to 107th wherein the second parameter is the temperature inside each package (2).

In a 110th aspect according to any one of the aspects from the 81st to 107th wherein the second parameter is the concentration of a predetermined gas or substance inside the package (2).

In an 111th aspect according to any one of the aspects from the 81st to 107th wherein the second parameter is the variation of concentration of a predetermined gas or substance within the package.

In a 112th aspect according to any one of the aspects from the 81st to 107th wherein the second parameter is the degree of acidity present inside the package (2) or the variation of the degree of acidity inside each package (2).

In a 113th aspect according to any one of the aspects from the 81st to 112th the infrastructure comprises the second level sensors (5).

In a 114th aspect according to any one of the aspects from the 81st to 113th each of the second level sensors (5) associated with a respective package comprises:
a component sensitive to temperature or a parameter proportional to temperature carried by the package of the product.

In a 115th aspect according to any one of the aspects from the 81st to 114th each of the second level sensors (5) associated with a respective package comprises:
a component sensitive to temperature or a parameter proportional to temperature inside the package. In a 116th aspect according to any one of the aspects from the 81st to 115th each of the second level sensors (5) associated with a respective package comprises:
a component, sensitive to the concentration or change in concentration of a predetermined gas, inserted inside the package.

In a 117th aspect according to any one of the aspects from the 81st to 116th each of the second level sensors (5) associated with a respective package comprises:
a component, sensitive to the concentration of a given substance or to the variation of a given substance, inserted inside the packaging.

In a 118th aspect according to any one of the aspects from the 81st to 117th each of the second level sensors (5) associated with a respective package comprises:
a component, sensitive to the concentration or the concentration variation of a predetermined gas, carried by the package.

In a 119th aspect according to any one of the aspects from the 81st to 118th each of the second level sensors (5) associated with a respective package comprises:
a component, sensitive to the concentration of a predetermined substance or to the variation of a predetermined substance, carried by the package.

In a 120th aspect according to any one of the aspects from the 81st to 119th each of the second level sensors (5) associated with a respective package comprises:
a component that is sensitive to the pH or pH variation of the environment inside the package.

In a 121st aspect according to any one of the aspects from the 81st to 120th each of the second level sensors (5) associated with a respective package comprises is a disposable component applied to the package (2) or inserted into the package.

In a 122nd aspect according to any one of the aspects from the 81st to 121st each of the second level sensors (5) associated with a respective package is a component printed directly on the package (2), optionally on a plastic film or on a plastic or paper tray which is part of the package (2).

In a 123rd aspect according to any one of the aspects the 81st to 122nd each of the second level sensors (5) is configured for providing:
an indication, optionally visual, of yes/no type of the fact that the product inside the respective package has exceeded or not a certain temperature for a specific time interval.

In a 124th aspect according to any one of the aspects from the 81st to 123rd each of the second level sensors (5) is configured for providing:
an indication, optionally visual, proportional to the maximum temperature reached by the product inside its packaging.

In a 125th aspect according to any one of the aspects from the 81st to 124th each of the second level sensors (5) is configured for providing:
an indication, optionally visual, of yes/no type of the fact that the area of the package where the second level sensor is arranged has exceeded or not a certain temperature for a specific time interval. In a 126th aspect according to any one of the aspects from the 81st to 125th each of the second level sensors (5) is configured for providing:
an indication, optionally visual, proportional to the maximum temperature reached in the area of the package where the second level sensor is present.

In a 127th aspect according to any one of the aspects from the 81st to 126 each of the second level sensors (5) is configured for providing:
an indication, optionally visual, of yes/no type of the fact that a certain concentration or concentration variation of a predetermined gas or gas mixture is exceeded within the respective package for a specific time interval.

In a 128th aspect according to any one of the aspects from the 81st to 127th each of the second level sensors (5) is configured for providing:
an indication, optionally visual, proportional to the maximum concentration of a predetermined gas or mixture of gases reached inside the respective packaging.

In a 129th aspect according to any one of the aspects from the 81st to 128th each of the second level sensors (5) is configured for providing:
an indication, optionally visual, of yes/no type of the fact that a certain concentration or concentration variation of a given substance or group of substances is exceeded inside the respective package for a specific time interval.

In a 130th aspect according to any one of the aspects from the 81st to 129th each of the second level sensors (5) is configured for providing:
an indication, optionally visual, proportional to the maximum concentration of a predetermined substance or group of substances inside the respective packaging.

In a 131st aspect according to any one of the aspects from the 81st to 130th each of the second level sensors (5) is configured for providing:
an optionally visual yes/no indication of whether or not a certain concentration or concentration change of a predetermined gas or gas mixture is exceeded at a surface of the respective packaging for a given time.

In a 132nd aspect according to any one of the aspects from the 81st to 131st each of the second level sensors (5) is configured for providing:
an indication, optionally visual, proportional to the maximum concentration of a predetermined gas or mixture of gases reached at one surface of the respective package.

In a 133rd aspect according to any one of the aspects from the 81st to 132nd each of the second level sensors (5) is configured for providing:
an indication, optionally visual, of yes/no type of the fact that inside the respective package a certain pH or pH variation has been exceeded or not exceeded for a specific time interval.

In a 134th aspect according to any one of the aspects from the 81st to 133rd each of the second level sensors (5) is configured for providing:
an indication, optionally visual, proportional to the maximum or minimum pH reached inside the package.

In a 135th aspect according to any one of the aspects from the 81st to 134th the second sensors include more than one sensor of different types.

In a 136th aspect according to any one of the aspects from the 81st to 135th the first sensors include more than one sensor of different types.

In a 137th aspect according to any one of the aspects from the 83rd to 136th the predetermined second level acceptability criterion is considered to be complied with for a given subgroup only if the indication, optionally visual, provided by each of the second level sensors (5) used in the second level procedure for that subgroup meets a respective verification test.

In a 138th aspect according to any of the aspects from the 83rd to 137th the step of the second level procedure of establishing which subgroups comply with a predetermined second level acceptability criterion includes:
receiving an identification, for each subgroup (13), of a predetermined number of sample packages (12), said sample packages (12) in a number lower than the total number of packages in each subgroup (13);
establishing that a subgroup complies with the predetermined second level acceptability criterion by only using the second level sensors (5) associated with the sample packages (12), or the products contained in the sample packages (12), present in the respective subgroup (13).

In a 139th aspect according to the preceding aspect said sample packages are in number lower than half, optionally lower than 25%, of the packages (2) present in each subgroup (13).

In a 140th aspect according to any one of the two preceding aspects the sample packages (12) of each subgroup (13) are placed in an outer peripheral area of the subgroup itself.

In a 141st aspect according to any one of the three preceding aspects when each subgroup (13) has packages (2) adjacent and superimposed with each other to form an ideal cube or parallelogram, each of the sample packages (12) is placed at a respective edge or vertex of the ideal cube or parallelogram.

In a 142nd aspect according to any of the preceding four aspects the sample packages (12) are two or more, more optionally from 3 to 6 for each subgroup (13).

In a 143rd aspect according to any one of the aspects from the 83rd to 142nd each package (2) is associated with a unique identification code.

In a 144th aspect according to any one of the aspects from the 83rd to 143rd each of the second sensors (5) of each package (2) is associated with a unique identification code.

In a 145th aspect according to any one of the aspects from the 83rd to 144th wherein the step of establishing which subgroups comply with a predetermined second level acceptability criterion comprises, for each subgroup:
receiving directly, or from said detector (8), at least one indication related to the second parameter, emitted or displayed by each of the queried or observed second sensors (5),
receiving, in association with each indication, the respective unique identification code of the respective package (2) of each of queried or observed the second sensors (5).

In a 146th aspect according to any one of the preceding three aspects the control supervisor (6) or the detector (8) is configured to identify in each subgroup (13) the sample packages (12) using an interrogation protocol comprising a list of unique package identification codes (2), and wherein the control supervisor (6) or the detector (8) is configured for selecting as sample packages (12) those belonging to the list present in the interrogation protocol.

In a 147th aspect according to any one of the preceding four aspects, the second level procedure comprises a step of verifying that the sample packages (12) identified in each subgroup (13) are all and only those present in a list of the query protocol including the unique identification codes of the sample packages (12) to be selected.

In a 148th aspect according to the preceding aspect the control supervisor (6) or detector (8) is configured for carrying out the verification phase.

In a 149th aspect according to one of the two preceding aspects the query protocol is stored in the control supervisor (6).

In a 150th aspect according to any one of the aspects from the 83rd to 149th said/one indication provided by each of the second level sensors (5) is read by or from a detector (8) communicatively connected to the control supervisor (6) and is transmitted to the latter.

In a 151st aspect according to the preceding aspect the detector includes:
at least an optical scanner or camera if the indication is of visual type, or
at least one radio wave detector if the indication is of electromagnetic type, or
at least one acoustic detector, if the indication is of sound type.

In a 152nd aspect according to any one of the aspects from the 81st to 151st the control supervisor comprises one or more digital computers and/or one or more analogue computers.

In a 153rd aspect according to any one of the aspects from the 81st to 152nd, the control supervisor (6) is configured for communicating to a distributed register (16) of blockchain type connected to said control supervisor (6), the following data:
the unique identification code for a first level sensor (4).

In a 154th aspect according to any one of the aspects from the 81st to 153rd the control supervisor (6) is configured for communicating to a distributed register (16) of blockchain type connected to said control supervisor (6), the following data:
the pair of values, consisting of the value of the first parameter and the sampling instant, detected by the first level sensor (4) during the execution of the first level procedure.

In a 155th aspect according to any one of the aspects from the 81st to 154th the control supervisor (6) is configured for communicating to a distributed register (16) of blockchain type connected to said control supervisor (6), one or more of the following data:
the unique identification code of a second level sensor (5).

In a 156th aspect according to any one of the aspects from the 81st to 155th the control supervisor (6) is configured for communicating to a distributed register (16) of blockchain type connected to said control supervisor (6), one or more of the following data:
the pair of values, consisting of the indication relative to the second parameter and the detection instant, recorded by the detector (8).

In an 157° according to any one of the four preceding aspects wherein the control supervisor (6) is configured for communicating said data periodically or at regular intervals or following a command to the distributed registry (16) of blockchain type connected to said control supervisor (6).

In a 158th aspect according to any one of the aspects from the 81st to 157th, the control supervisor (6) is configured for certifying that one or more subgroups (13) present in the load volume (3) are only formed of products complying with a predetermined quality standard if, in a reference time interval during which the first level procedure is executed a plurality of times, the following occurs:
the values of the first parameter comply with the predetermined first level acceptability criterion each time the first level procedure is executed.

In a 159th aspect according to any one of the aspects from the 81st to 158th, the control supervisor (6) is configured for certifying that one or more subgroups (13) present in the load volume (3) are formed only of products that comply with a predetermined quality standard if, in a reference time interval during which the first level procedure is performed a plurality of times, the following occurs:
the values of the first parameter do not comply with the predetermined first level acceptability criterion during at least one execution of the first level procedure, and
when the second level procedure is executed, it is verified that all said one or more subgroups (13) in the load volume comply with the predetermined second level acceptability criterion.

In a 160th aspect according to one of the two preceding aspects where the reference time interval is equal to the duration of one transport of the load from a delivery point to a destination.

In a 161st aspect according to any one of the aspects from the 83rd to 160th each of the second level sensors (5) associated with a respective package (2) is
a disposable component attached to or inserted inside the packaging (2).

In a 162nd aspect according to any one of the aspects from the 83rd to 161st each of the second level sensors (5) associated with a respective package (2) is
a component printed directly on the package (2), optionally on a plastic film or on a plastic or paper tray forming part of the package (2).

In a 163rd aspect according to any one of the aspects from the 81st to 162nd the load volume (3) of the infrastructure (1) is defined within a loading area of a land vehicle, ship, or aircraft.

A 164th aspect concerns an infrastructure according to any one of the aspects from the 81st to 163rd configured for implementing the process according to any one of the aspects from the 1st to 80th.

A 165th aspect concerns also a computer implemented process according to any one of the aspects from the 1st to 80th, for example executed by the infrastructure according to any one of the aspects from the 81st to 163rd

A 166th aspect concerns a software product comprising instructions which when executed by a programmable control unit configures said programmable control unit to execute a process according to any one of the aspects from the 1st to 80th.

In a 167th aspect according to the preceding aspect the instructions are stored in a memory.

In a 168th aspect according to the preceding aspect the memory comprises an optical or magnetic disk memory or an EPROM-type memory or a volatile memory.

In a 169th aspect according to any one of the preceding three aspects the instructions are carried by an electromagnetic signal, optionally in a data package transmitted via the Internet.

### BRIEF DESCRIPTION OF THE DRAWINGS

Some embodiments and aspects of the invention will be described below with reference to the accompanying drawings, provided solely for illustrative purposes and therefore not-limiting purposes: wherein:

FIG. 1 is a block diagram relative to an infrastructure for monitoring a load of perishable products in accordance with the present invention;

FIG. 2 is a perspective view relating to a subgroup (e.g., a pallet) formed by a plurality of packages; the subgroup or pallet is housed within a loading bay served by the infrastructure according to the present invention;

FIGS. 3-5 are schematic views of respective embodiments of an infrastructure according to the present invention;

FIG. 6 shows a flow chart of the steps of a process performed by an infrastructure in accordance with the present invention, in particular performed by the infrastructures of FIGS. 3-5.

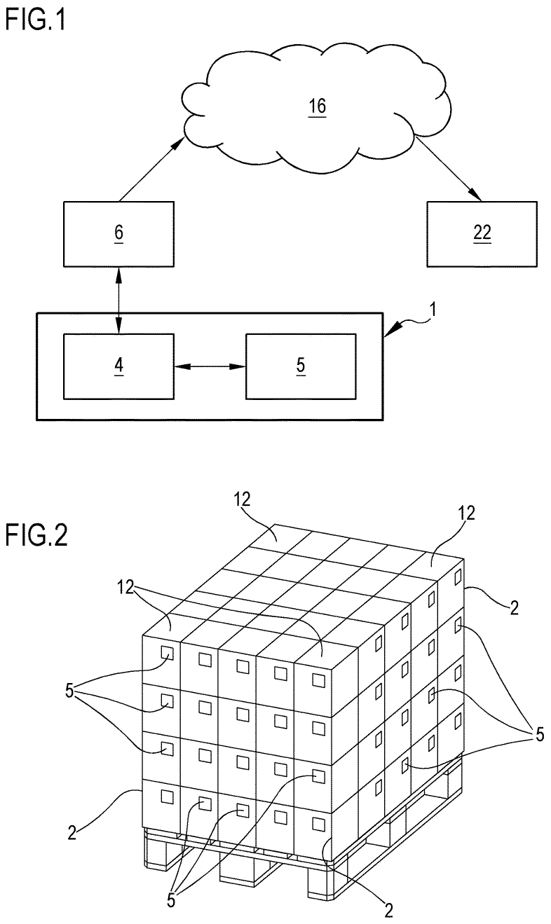

### CONTROL UNIT

The process and infrastructure for monitoring a load of perishable products described and claimed herein, respectively, make use of or comprise at least one control supervisor 6 and any further control units in charge of receiving signals from various sensors and performing one or more monitoring steps described and/or claimed.

The control unit in the supervisor 6 may be a single unit or may be formed by a plurality of distinct control units depending on the design choices and operational requirements.

N.B.: ‘Circuit element’ is a single active or passive functional part of an electronic circuit, such as one diode, one transistor, one resistor, one capacitor, etc. The term ‘control unit’ means an electronic component which may comprise at least one of: a digital processor (CPU), an analogue circuit, or a combination of one or more digital processors with one or more analogue circuits. The control unit may be “configured” or “programmed” to perform certain steps: this may be accomplished in practice by any means that allows the control unit to be configured or programmed. For example, in the case of a control unit comprising one or more CPUs and one or more memories, one or more programs may be stored in appropriate memory banks connected to the CPU(s); the program(s) contain instructions which, when executed by the CPU(s), program or configure the control unit to perform the operations described in relation to the control unit. Alternatively, if the control unit is or includes analogue type circuitry, then the circuitry of the control unit may be designed to include circuitry configured, in use, to process electrical signals such as to perform the steps related to the control unit.

### DETAILED DESCRIPTION

### Monitoring Infrastructure

The term 1 has been generally indicated to describe an infrastructure for monitoring a load of perishable products, such as foodstuffs, pharmaceutical products or other products that are sensitive to the environmental conditions prevailing in the environment in which the product is stored; in particular, the infrastructure 1 may be used for monitoring products stored at a storage site or for monitoring products during transport from a production or sorting site to a destination site. The monitoring infrastructure 1 allows values representative of the state of preservation of the transported products to be measured and stored permanently within a distributed registry, for example implementing blockchain technology. The monitoring infrastructure 1 may be used, for example, in the transport of fresh or frozen foodstuffs, so as to certify that an ideal temperature is maintained during transport and that the quality of the products transported is guaranteed. In fact, the infrastructure makes it possible to monitor and certify the maintenance of the cold chain during the transport of fresh or frozen products, the latter typically kept at temperatures below 0° C. The monitoring infrastructure may also enable the implementation of smart contracts between a party responsible for the transport of perishable products and a recipient, in order to unequivocally define the responsibilities of the parties.

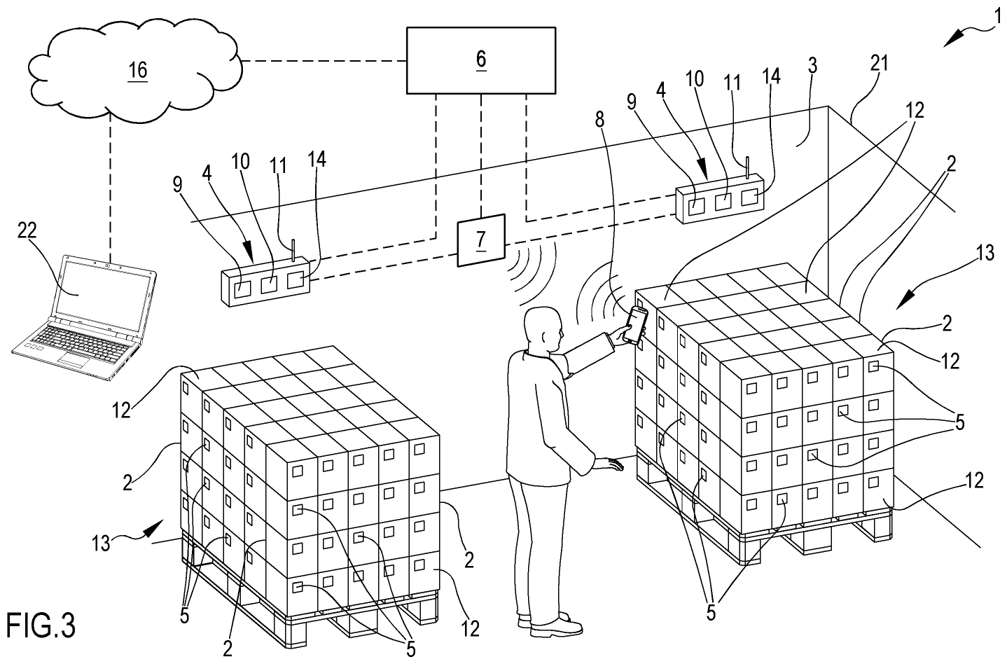

As for example visible in FIGS. 3-5, the monitoring infrastructure 1 may be used to monitor product packages present in a load compartment 21 defined by a storage unit or conveyor. The load compartment 21 delimits a predetermined load volume 3 configured to house a plurality of packages 2 each of which houses one or more products to be transported. Note that the load volume 3 may be defined within a load volume 3 of a land vehicle, a ship, or an aircraft. In order to enable the infrastructure 1 to perform monitoring, the packages 2 are organized into a plurality of distinct subgroups 13 of adjacent packages in the load volume. Each subgroup 13 comprises a plurality of packages adjacent and superimposed each other, for example in such a way as to define pallets of cubic or parallelogram shape (FIG. 2).

Referring now to the example embodiment of FIG. 3, the infrastructure 1 comprises one or more first level sensors 4, operating in the load volume 21, configured to detect a first parameter related to an environment present in the load volume 3. In detail, FIG. 3 involves the use of a plurality of first level sensors 4 distributed inside the load volume 3, such that they are sensitive to possible fluctuations of the first parameter in the various areas of the volume in which the sensors are installed. In fact, the load volume 3 may have a high spatial extension, making it preferable for the first parameter to be detected by the first level sensors at various positions within the load volume 3 itself. Each first level sensor 4 may comprise a memory 10 configured to store one or more values assumed by the first parameter, as well as to store a further parameter representative of the time instant at which each value of the first parameter is sampled. The memory 10 may further be configured to permanently store a predetermined unique identification code suitable for identifying each first level sensor 4 installed within the load volume 3.

The first level sensor 4 may further comprise a control unit 9 configured for executing a sampling of the first parameter and store, within the memory 10, the measured value of the first parameter and the value representative of the time instant at which sampling is performed. Note that the first parameter is representative of a temperature value or a parameter function of the temperature; or even of gases present in the environment or both of them. Note that specific gases are normally originated by products in their deterioration process (freshness markers), especially if the packaging used for the products is permeable or semi-permeable to gases. The first level sensor 4 may therefore comprise a temperature sensor or a sensor for the presence or concentration of a volatile substance, for example a gas sensor 14 configured to measure a temperature value within the load volume 3 at the position of the first level sensor 4. The first level sensor 4 is further configured to store within the memory 10, a pair of values, for example a temperature value or concentration of a gas/gas mixture present in the load volume 3 and an indicator of the sampling instant at which the temperature value or gas concentration is detected.

Below, in order not to make the discussion too heavy, we will describe the case in which the first parameter is the temperature, but as already mentioned, the concentration of a prefixed gas or gas mixture can alternatively be used as the first parameter and therefore what will be said below in relation to the temperature as the first parameter and the relative temperature values can be used, mutatis mutandis, apply to the case where the first parameter is the concentration or concentration variation measured by appropriate first sensors of a predetermined gas or gas mixture present in the environment of the load volume outside the packages. Each first level sensor 4 may further comprise at least one transmitter 11 configured to send the pair of temperature-sampling instant values to a control supervisor 6, the latter detailed in the following. In particular, the transmitter 11 may for example be at least one of a Bluetooth transmitter, a WiFi transmitter, an RFID transponder tag, an NFC device. The transmitter 11 may be configured to send to the control supervisor 6 a package of information comprising the unique identification code of the first level sensor 4 and the pair of temperature values—sampling instant. The predetermined unique identification code allows each first level sensor 4 to be differentiated and consequently to associate each pair of temperature values—sampling instant with the first level sensor 4 that made the measurement.

The infrastructure may further comprise second level sensors 5: in particular, each second level sensor is associated with a respective package 2 and configured to detect an alteration of a second parameter relative to an environment adjacent to or inside each package 2. Each second level sensor 5 and thus each package are identifiable by a unique identification code suitable for distinguishing the packages 2 from each other. Each second level sensor comprises at least one component sensitive to the variation of the second parameter, the latter relative to a temperature value, or a parameter function of the temperature, of each package 2 or of an environment within each package. It should therefore be noted that the second level sensors may be suitable for detecting the instantaneous value of the measured parameter, or simply be sensitive to a variation or exceeding of a threshold relative to the parameter itself. In detail, each second level sensor 5 used in the example of FIG. 3 is suitable for sensing a variation of temperature inside or outside the package or a variation of another parameter providing the perishable state of the food product. In fact, the second level sensor 5 may be a sensor sensitive to:
a temperature or parameter function of the temperature, carried on or in the product package,
a component sensitive to the concentration of a predetermined gas inserted inside the package, or even outside in the case of permeable or semi-permeable packaging, or
a pH-sensitive component of the product or other biochemical markers on its surface or in the environment inside the package.

Depending on the type of product, sensors sensitive to the most appropriate parameter can be used to indicate the state of deterioration of the product.

Since a large number of packages are present inside the load volume 3, and therefore a large number of second level sensors 5 (each of which is associated with a respective package 2), in order to keep a low cost of each package, the second level sensors 5 may be components of simple construction, for example also of the disposable type, and therefore of a different type than the first level sensors 4.

In particular, the second level sensors shown in FIG. 3 are applicable to the package 2, or can be inserted inside the package itself, or can be printed directly on the package 2 (for example on a plastic film or on a tray forming part of the package 2 itself).

Each second level sensor 5 can be configured to provide at least one between:
a boolean indication of the fact that the product within the respective package 2 has exceeded or not a certain temperature for a specific time interval;
an indication proportional to the maximum temperature reached by the product inside the respective package 2;
a boolean indication of the fact that the area of the package 2 where the second level sensor 5 is present has exceeded or not a certain temperature for a specific time interval;
an indication proportional to the maximum temperature reached by an area of the package 2 associated with the second level sensor 5;
a boolean indication of the fact that the product inside the respective package 2 has exceeded or not a certain concentration of a predetermined gas over a specific time interval;
an indication proportional to the maximum concentration of a predetermined gas reached by the product inside the respective packaging 2;
a boolean indication of the fact that inside a respective package 2 a certain pH has been exceeded or not for a specific time interval;
an indication proportional to the maximum or minimum pH reached inside the respective package 2;
an indication that another indirect or direct marker of micro-organism growth, e.g. spoilage and/or pathogens, has reached a threshold within the package 2.

In practice, in the example of FIG. 3 each second level sensor 5 may be a passive sensor, unable to make measurements of absolute values of a parameter and storing the data obtained, but simply capable of providing an indication, for example visual, or a signal resulting from the detection of a change above a threshold of the monitored parameter. The second level sensor 5 may, for example, be a fluorimetric or colorimetric sensor capable of changing the coloration (or other visually appreciable property) of a surface of the same sensor as a function of temperature, so as to give evidence of a detection of a temperature (or a change thereof) on or in the package above a predetermined threshold. Alternatively, the second level sensor 5 may modify a graphical portion, which may for example comprise a QR code or barcode, so as to identify the exceeding of a threshold temperature or a predetermined thermal variation. In a further variant, the second level sensor 5 may comprise a magnetic ink print, made on the tray or film of the package: when subject to a magnetic field, the sensor is able to emit a magnetic signal proportional to the temperature, which thus allows indirect measurement of the temperature value on or in the package.

Although the parameter monitored in the context of food products is often the temperature, the possibility of monitoring parameters such as the concentration of a substance or gas or the degree of acidity inside the package with second level sensors is not excluded (as already mentioned), preferably using passive elements of simple design that can provide an indication or signal related to the monitored parameter.

The infrastructure of FIG. 3 comprises at least one detector 8 configured to read and/or receive information emitted by the second level sensors 5. The detector 8 may be a dedicated device for reading the information emitted by the second level sensors 5, such as for example an optical scanner or a camera, an electromagnetic wave detector or an acoustic detector. However, it is not excluded that the detector 8 may comprise an application (in particular software program) installable on a general purpose device, such as a smartphone or tablet, capable when executed of configuring the general purpose device to read or receive the indications and/or signals displayed and/or emitted by the second level sensors.

The detector(s) 8 is (are) communicatively connected to the first level sensors 4 (which in this case act as intermediaries) or directly connected to the control supervisor 6.

The detector 8 can effectively detect and analyze the information generated/visualized by the second level sensors, store it in a local memory and transmit it to the control supervisor 6.

In accordance with the first embodiment of the invention shown in FIG. 3, the detector 8 is a device manually operated by an operator interrogating the second level sensors 5. The analog information (for example, visual, magnetic or acoustic) emitted/shown by the second level sensors is, via the detector 8, transformed into information, for example digital, that can be transmitted to one or more other components of the infrastructure.

The detector 8 may further have a user interface suitable for indicating to the person in charge, the unique identification code of the second level sensors 5 to be interrogated. The first level sensors 4 or the control supervisor 6, can be configured to communicate to the detector 8, the reading of predetermined second level sensors 5 according to a predetermined criterion, in such a way as to optimize the timing of reading the information as well as to impose the sensors to be consulted, useful to certify the quality of the entire pallet without the need to interrogate all the second level sensors of the pallet itself.

The infrastructure may further comprise an intermediate transmission unit 7 communicatively interposed between the first level sensors 4 and the control supervisor 6. In detail, the intermediate transmission unit 7 may be defined by a digital and/or an analogue processing unit suitable for defining a data transmission channel between the first level sensors 4 and the control supervisor 6. The intermediate transmission unit 7 may for example comprise a router, suitable for defining a local network configured for putting each first level sensor 4 into communication with the control supervisor 6, or to define communication between the latter via the internet network.

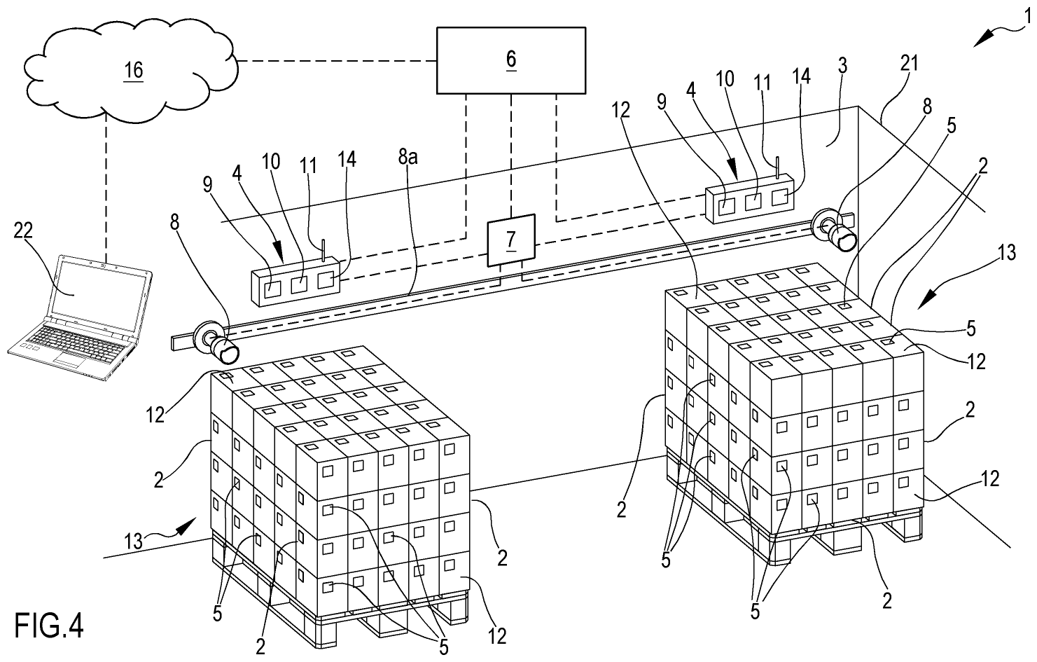

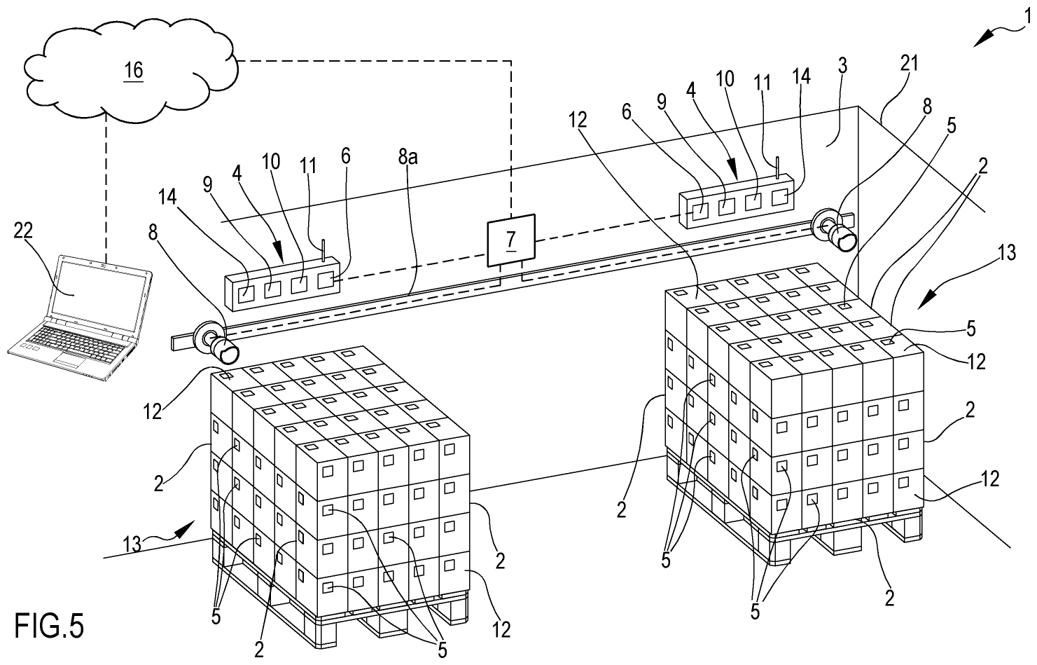

As previously mentioned, the infrastructure may comprise at least one control supervisor 6 formed by a digital and/or analog processing unit directly or indirectly connected to the first level sensors 4, the detector 8 and possibly also to the second level sensors 5. The control supervisor is configured for commanding the execution of the process that will be described later and to receive from the first sensors the values of the first parameter and from the detector or the second sensors the indications/values relative to the second parameter. In other words, the control supervisor 6 can be defined as a control unit suitable for coordinating the first level sensors 4 and the detector 8, respectively for measuring the first and the second parameter. From a structural point of view (FIG. 3 and FIG. 4), the control supervisor 6 is arranged in a remote position with respect to the load volume 3. However, it is not excluded that the control supervisor 6 is arranged inside the load volume 3, or included within each first level sensor 4 (as will be illustrated in the example in accordance with the third embodiment of the infrastructure shown in FIG. 5).

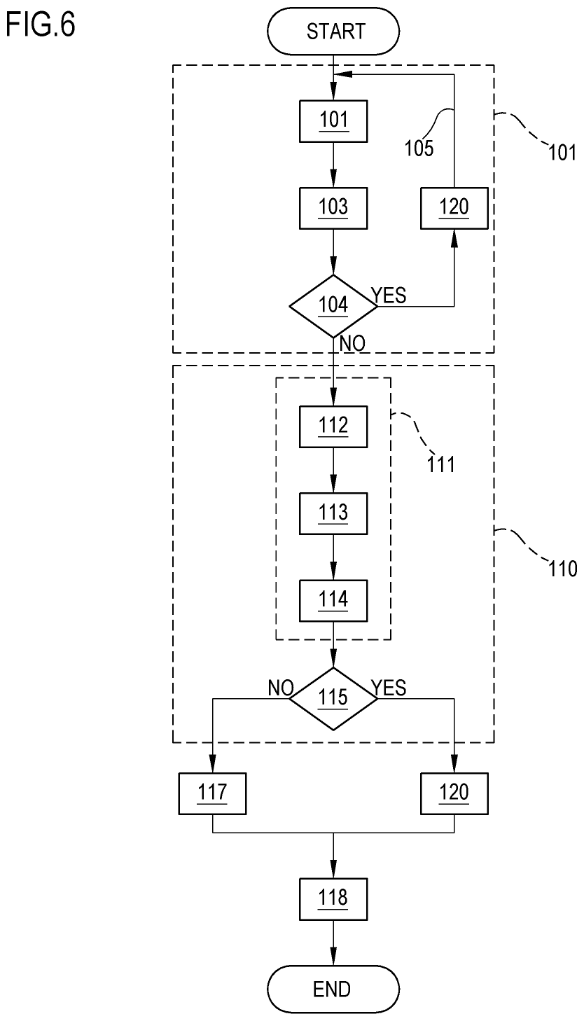

The control supervisor 6 is configured to execute a monitoring process 100 (see FIG. 6) comprising the execution of a first level procedure 101 and a subordinate second level procedure 110 which act in cascade upon the occurrence of the conditions described below. In particular, the monitoring supervisor 6 is configured to primarily execute the first level procedure 101 comprising the steps of:
commanding each first level sensor 4 to sample the first parameter (step 102),
receiving, or determine, on the basis of the signals emitted by each first level sensor 4, the values assumed by the first parameter (step 103),
determining if the values of the first parameter comply or not with a predetermined first level acceptability criterion (step 104).

If, as a result of the query referred to in step 104, it is determined by the control supervisor 6 that the values of the first parameter comply with the predetermined first level acceptability criterion, the procedure comprises cyclically repeating the execution of the first level procedure (see recirculation line 105 in FIG. 6). For example, the supervisor may be configured to repeat the execution of steps 102-104 periodically (for example after a time interval of one or more minutes has elapsed), or upon command of an input given by an operator suitable for communicating with the supervisor, or following the occurrence of a predetermined event (for example the variation of an external parameter, the running of a predetermined kilometric route or other event), or at regular intervals given by a predetermined rule managed by the supervisor (for example the repetition of the cycle may occur more frequently as time goes by).

As previously mentioned, the control supervisor 6 is configured to receive from each first level sensor 4 the aforementioned package of information (for example comprising the unique identification code of the first level sensor 4 and the pair temperature value sampling time instant), so as to determine whether the sampled first parameter values comply with the first level acceptability criterion or not (step 104). In fact, the comparison with the first level acceptability criterion may comprise comparing each first parameter value sampled by each sensor 4 with a range of values deemed acceptable, for example a range of temperatures, or a range of parameter values as a function of temperature.

If the first level acceptability criterion is not complied with, the control supervisor 6 is configured to perform the second level procedure (step 110). In particular, the supervisor may connect communicatively with the detector 8, so as to receive information relative to the second parameter provided by the second level sensors 5.

In particular, the second level procedure (step 110) allows to identify and report subgroups 13 of packages 2 that do not comply with a prefixed second level acceptability criterion on the basis of the information provided by the second level sensors 5. In particular, the prefixed second level acceptability criterion is considered to be complied with for a given subgroup 13, only if the information provided by each of the second level sensors 5 belonging to the subgroup under examination, responds to a respective verification test.

The second level procedure thus allows to determine which subgroups 13 comply with a predetermined second level acceptability criterion. In particular, the second level procedure allows to identify for each subgroup 13, a prefixed number of sample packages 12, as well as to establish if a subgroup 13 complies or not with the predetermined second level acceptability criterion exclusively using the information emitted by the second level sensors 5 exclusively associated with the sample packages 12. In this regard, it should be noted that each subgroup 13 belongs to a predetermined number of sample packages 12, which are smaller in number than the total number of packages of the subgroup 13 to which it belongs. In particular, the sample packages 12 are preferably the packages placed in an outer peripheral area of the subgroup itself, for example at a respective edge or a respective upper or lower vertex of the pallet.

It should be noted that the analysis of the sample 12 packages alone minimizes the time required to carry out the second level procedure and reduces the amount of information collected to manage the second level verification test. As a matter of fact, the packages 2 placed at the top of the pallet are the packages most subject to heat loss (or to the action of external agents of any nature), the analysis of the sample packages 12 only allows therefore to measure the temperature value or to detect a temperature variation (or other parameter) in correspondence with the packages that have been subject the greatest thermal change (or change in other monitored parameter). In other words, verifying that the temperature readings (or other parameter) provided by the second level sensors 5 associated with the sample packages 12 comply with the predetermined second level acceptability criterion ensures that the predetermined second level acceptability criterion is also complied with for the untested packages.

More in detail, still referring to the flow chart of FIG. 6, the second level procedure involves identifying in each subgroup 13 the sample packages 12 to be analyzed by executing an interrogation protocol (step 111). In particular, the control supervisor 6 is configured to select, as sample packages 12, those belonging to a list of unique identification codes of the packages 2 present in the interrogation protocol (step 112), for example corresponding to the packages 2 arranged at the vertices of the pallet.

The control supervisor 6 is then configured to send the command signal to each of the detectors 8 to command the latter to read the second level sensors according to the interrogation protocol (always step 112). The control supervisor 6 is further configured to receive directly from the second level sensors or the detector 8 the indication relative to the second parameter detected by the second sensors 5, as well as to receive the respective unique identification code of the respective package 2 of each of the queried second sensors 5 (step 113). The control supervisor 6 is configured to verify that the sample packages 12 identified in each subgroup 13 have an identification code that is the same as the sample packages 12 in the interrogation protocol list (step 114). If there is a match between said identification codes, the data is considered valid, otherwise the control supervisor 6 sends a new command signal to the detector 8 to request the re-reading of a second level sensor 5 present in the interrogation protocol.

Following receipt of the information sampled by the second level sensors 5 and verification that this information has been recorded by the second level sensors 5 belonging to the querying protocol, the control supervisor 6 is configured for executing the verification test to determine whether the data sampled by the second level sensors comply with the second level acceptability criterion (step 115). As discussed above, the supervisor 6 is then configured for comparing each information/value of the second sampled parameter with a respective acceptability criterion (part of step 115), for example in case the parameter of interest is temperature with an acceptable temperature range, or a reference (chromatic, acoustic, or other nature) deemed acceptable. Depending on the information detected by the first and second level sensors 4, 5, the control supervisor 6 is configured for certifying that one or more subgroups 13 present in the load volume 3 are formed exclusively of products that comply with a predetermined quality standard (i.e. from a level of quality deemed acceptable). In other words, for example during transport of the load from a delivery point to a destination, the control supervisor 6 certifies the acceptable quality of all products transported (step 120) if, in a reference time interval during which the first level procedure is performed a plurality of times, the following is verified:
the values of the first parameter comply with the predetermined first level acceptability criterion each time the first level procedure is performed, or if
the values of the first parameter do not comply with the predetermined first level acceptability criterion during at least one execution of the first level procedure, and
when the second level procedure is performed, it is verified that said one or more subgroups (13) comply with the predetermined second level acceptability criterion.

In practice, the reference time interval may coincide with the duration of a load transport from a delivery point to a destination: during this interval the monitoring process is carried out by repeating the first level procedure several times. If the first level acceptability criterion is always complied with each time the first level procedure is performed, it goes without saying that the quality can be certified without the need to perform the second level procedure. If, on the contrary, one or more times the first level procedure is not positively passed (first acceptability criterion not satisfied), the second level procedure is activated: after the execution of the second level procedure for each subgroup or pallet, the compliance with the second acceptability criterion is verified for all the subgroups present in load volume 3 (step 115). If the second acceptability criterion is complied with for all the subgroups, the monitoring process requires the inspection supervisor to certify the acceptable quality of all the products present in the loading volume itself (step 120). The control supervisor is configured for emitting a signal indicating that one or more subgroups or pallets do not comply with the intended acceptability criterion (step 117) and therefore it is not possible for those subgroups to certify acceptable quality.

The supervisor 6 is further configured to communicate periodically or at regular intervals or following a command or following the detection of an event, to a blockchain-type distributed registry 16 connected to the control supervisor 6, at least one (preferably all) of the following parameters (step 118):
the unique identification code relating to each first level sensor 4 operating in load volume 3,
the pair temperature values (or value of other first parameter detected by the first level sensors)-sampling time instant measured by each first level sensor 4 operating in the load volume 3 during the execution of the first level procedure,
the unique identification code of a second level sensor 5 operating in load volume 3,
the pair temperature value (or other indication given by the second level sensors)-detection time instant recorded by the detector 8.

Each of the data detected and/or measured by the first and second level sensors 4, 5, whether or not they respectively comply with the first and second level acceptability criteria, are available to a user of the infrastructure exclusively following access to the distributed register via a user device connected to a local area network or to an internet network. The infrastructure allows a user to download each of the data respectively measured and/or detected by the first level sensor 4 and the detector 8, so as to recreate the thermal history (or the history of any other parameter or set of parameters) of the transported products. The use of the blockchain technology allows to give evidence to the user of possible tampering of the temperature data and/or sampling instants stored in the distributed register, guaranteeing the genuineness of the detected and stored data and consequently of the transported product.

The infrastructure may further comprise at least one emitter 22 of a visual and/or acoustic signal arranged at the side of the load compartment or remotely, connected to the distributed register 16 and configured for giving evidence to a user of non-compliance with the second level acceptability criterion for a predetermined time interval. Note that the emitter 22, in an embodiment of the invention is an application for example installable on a smartphone or a PC, suitable for notifying a subject receiving the load, of non-compliance with the second level acceptability criterion.

In the embodiment exemplified in FIG. 4, an infrastructure 1 is schematically illustrated having most of the structural and functional characteristics described above and also suitable for executing the monitoring process schematically illustrated in FIG. 6. In order to avoid repetitions, in FIG. 4 the components already described with reference to the example of FIG. 3 have been represented with the same numerical references. In particular, the second embodiment shown in FIG. 4, provides that the detector 8 may comprise at least one of a camera and a radio wave detector, configured for automatically reading the information shown/supplied by the second level sensors 5. In particular, the detector 8 may have a plurality of cameras and/or radio wave detectors distributed throughout the load volume 3 and facing the packages 2, so as to easily read the information shown/supplied by the second sensors 5, digitize it and transmit it to the control supervisor 6 or to a first level sensor 4. Each camera or radio wave detector, may further be movable along rails 8a integral with the loading area 21, in such a way as to scan at least part of the load volume 3. Each detector 8 may further have a respective control unit connected to the first level sensors 4 and/or the control supervisor 6 and configured for receiving a command signal emitted by a first level sensor 4 or by the control supervisor 6, representative of the position inside the load volume 3, of the second level sensors 5 to be read. Each detector 8, following receipt of the command signal, is configured for moving relative to the load volume 3, along the guides 8a and position itself at a second level sensor 5 to be read. The detector 8 is then configured for transmitting to the first level sensor 4 or the supervisor 6 the detected information.

According to the latter embodiment of the invention, a reduced number of cameras and/or radio detectors allow for the reading of the second level 5 sensors in an automatic manner, thereby decreasing the manufacturing costs of the infrastructure.

In the example embodiment of FIG. 5, an infrastructure 1 having the structural and functional characteristics described above with reference to FIG. 4 and also suitable for executing the monitoring process schematically illustrated in FIG. 6 is schematically illustrated. In order to avoid repetitions, in FIG. 5 the components already described with reference to the embodiment of FIG. 3 and FIG. 4 have been represented with the same numerical references. The only difference with respect to the embodiment of FIG. 4 concerns the control supervisor 6 which in FIG. 5 is incorporated in one or more of the first level sensors. In this embodiment, therefore, the supervisor hosted by the first level sensor interacts with the other first level sensors and with the second level sensors by performing the process of FIG. 6.

The described and claimed process and infrastructure achieves one or more of the stated objectives and also guarantees a high degree of reliability through a redundancy of detection levels, which in each case is carried out with a criterion that minimizes the complexity of the system and the data load to be managed.

The process and infrastructure described and claimed herein may comprise further embodiments falling within the scope of protection of the accompanying claims. In particular, although the process described and claimed comprises the execution of a first level procedure (using the first level sensors) and a second level procedure (using the second level sensors), it may be contemplated that the process further comprises a third level procedure (or further levels of inspection) which is executed following the execution of the second level procedure to analyze even more extensively subgroups of subgroups (if, for example, dealing with loads involving a large number of products). Similarly, the infrastructure 1 may be configured for executing a process comprising a first level procedure (using the first level sensors) and a second level procedure (using the second level sensors) and a third level procedure (as well as further inspection levels) which is executed following the execution of the second level procedure to analyze even more capillary subgroups of subgroups (if, for example, dealing with loads involving a large number of products). In addition, each of the first level sensors may comprise several sensors combining, in a single unit suitable for providing the reading of various first level parameters, the different types of first level sensors provided above. Similarly, each of the second level sensors may comprise multiple sensors combining the different types of second level sensors provided above thus being able to provide various types of information for each package.

Finally, from a physical point of view, the control supervisor, as already mentioned, may be a remote unit with respect to the load compartment or alternatively it may include one or more units in the load compartment. In particular (according to the desired implementation architecture) the control supervisor may partly comprise elements arranged in the load compartment and partly elements remote from the same: in fact the control supervisor, performing calculation functions, may be centralized or not and therefore located entirely or partly in the load compartment or in proximity thereto (for example on board the means for transportation where the load compartment is located). In a particular alternative, part of the supervisor's computing functions could be incorporated into the detector or into a smartphone or tablet or other portable device that also includes the detector 8.

1.-37. (canceled)

38. A system for monitoring a load of perishable products, the load comprising a plurality of products in respective packages, the load being located in a load volume of a storage unit or of a conveyor,
wherein:
the load volume comprises a first level sensor configured to detect a first parameter relative to an environment of the load volume,
the load volume comprises subgroups of the plurality of products present in the load volume, wherein each of the subgroups comprises products which represent a fraction of the plurality of products in the load volume,
each of the respective packages is associated with a second level sensor configured to sense an alteration of a second parameter relative to an environment adjacent to or inside of the respective package;
the system comprises a controller configured to execute a first level procedure, wherein, during execution of the first level procedure, the controller causes the system to:
determine, based on signals emitted by the first level sensor, values of the first parameter over time based on detections of the first parameter by the first level sensor,
determine whether the values of the first parameter comply with a predetermined first level acceptability criterion, and
if the values of the first parameter comply with the predetermined first level acceptability criterion, repeat the execution of the first level procedure;
wherein the controller is further configured to execute a second level procedure if the values of the first parameter do not comply with the predetermined first level acceptability criterion, wherein, during execution of the second level procedure, the controller causes the system to:
determine, based at least in part on indications relative to the alteration of the second parameter present in each respective subgroup detected by the second level sensor, which subgroups comply with a predetermined second level acceptability criterion.

39. The system of claim 38, wherein the first and second level sensors are different types of sensors;
wherein, during execution of the second level procedure, the controller is configured to:
communicate with the first level sensor directly or indirectly via an intermediate transmission unit (7) communicatively interposed between the controller and the first level sensor,
receive either the signals emitted by the first level sensor or the values of the first parameter over time,
communicate with the second level sensor directly or indirectly via a detector configured to read the indications emitted by the second level sensor, and
receive, from either directly from the second level sensor or indirectly via the detector, the indications relative to the alteration of the second parameter.

40. The system of claim 38, wherein the controller is configured to cause the system to certify that the products in the load volume comply with a predetermined quality standard if, in a reference time interval during which the first level procedure is executed a plurality of times, one or more of the following conditions are met:
the values of the first parameter comply with the predetermined first level acceptability criterion during each execution of the first level procedure,
the second level procedure is never executed, or
if the second level procedure is executed, the subgroups comply with the predetermined second level acceptability criterion.

41. The system of claim 38, wherein the first level sensors comprises a control unit (9), a memory (10) and a transmitter (11) that are communicatively coupled to each other either directly or indirectly, wherein the control unit is configured to cause:
the memory to store values assumed over time by the first parameter, and
the transmitter to transmit the values to the controller before the controller executes the first level procedure.

42. The system of claim 37, wherein the first parameter is detected at the position in the load volume in which the first level sensor operates and the first parameter is at least one of the following:
temperature,
a parameter that is a function of temperature,
a concentration of a predetermined gas or gas mixture, or
a variation of concentration of a predetermined gas or gas mixture,
wherein said predetermined first level acceptability criterion is respectively defined by at least one of:
a temperature interval,
an interval of values of the parameter that is a function of the temperature,
a concentration interval of a predetermined gas or gas mixture, or
an interval in the concentration variation of a predetermined gas or gas mixture.

43. The system of claim 37 wherein the first level sensor is one of a plurality of first level sensors that are distributed in the load volume, wherein each of the plurality of first level sensors is associated with a unique identification code;
wherein each of the plurality of first level sensors is configured to transmit, to the controller, values assumed over time of the first parameter with the unique identification code associating the respective one of the plurality of first sensors, and
wherein the controller is configured to identify the values assumed over time of the first parameter detected by each of the plurality of first sensors.

44. The system of claim 37, wherein the second parameter is at least one of the following:
temperature of an environment inside the package,
a parameter that is a function of temperature of an environment inside the package, temperature of an external surface of the package,
a parameter that is a function of the temperature of an external surface of the package, a concentration of a predetermined gas or of a predetermined substance inside the package,
a concentration variation of a predetermined gas or substance inside the package,
a concentration of a predetermined gas or of a predetermined substance on an external surface of the package,
a concentration variation of a predetermined gas or substance on an external surface of the package,
a degree of acidity present inside the package, or
a variation of the degree of acidity inside the package.

45. The system of claim 37, wherein the second level sensor comprises at least one of the following:
a component sensitive to a temperature or to a parameter proportional to a temperature carried by the package of the product,
a component sensitive to a temperature or to a parameter proportional to a temperature inserted inside the package,
a component inserted inside the package, wherein the component is sensitive to a concentration or to a concentration variation of a predetermined gas,
a component inserted inside the package, wherein the component is sensitive to a concentration of a predetermined substance or to a variation of a predetermined substance,
a component carried by the package, wherein the component is sensitive to a concentration or to a concentration variation of a predetermined gas,
a component carried by the package, wherein the component is sensitive to a concentration of a predetermined substance or to a variation of a predetermined substance, or
a component sensitive to a pH level or to pH level variation of the environment inside the package.

46. The system of claim 37, wherein during execution of the second level procedure, the controller is configured to cause the system to determine which subgroups comply with the predetermined second level acceptability criterion by:
receiving an identification, for each subgroup, of a predetermined number of sample packages, wherein a number of the sample packages is lower than a total number of packages in each subgroup, and
establishing that a subgroup complies with the predetermined second level acceptability criterion based on a plurality of second level sensors that are associated with the sample packages, or with products contained in the sample packages, present in the respective subgroup;
wherein the sample packages of each subgroup are placed in an external peripheral zone of the subgroup.

47. The system of claim 46, wherein the controller or the detector is configured to identify, in each subgroup, the sample packages based on an interrogation protocol that comprises a list of unique identification codes of the packages, and wherein the controller or the detector is configured to select as sample packages among those belonging to the list present in the interrogation protocol.

48. A computer-implemented method of monitoring a load of perishable products, wherein:
the load includes a plurality of products housed in respective packages and placed in a load volume,
the load volume a first-level sensor configured to detect a first parameter relative to an environment present in the load volume,
the load volume includes a plurality of subgroups of the plurality of products present in the load volume, wherein each subgroup includes a plurality of products which represent a fraction of the plurality of products present in the load volume,
each of the respective packages is associated with a second level sensor configured to sense an alteration of a second parameter relative to the respective package or to an environment inside of the respective package;
wherein the computer-implemented method comprises:
executing, by a computing device, a first level procedure comprising:
determining values assumed by the first parameter over time based on detections of the first parameter by the first level sensor,
establishing whether the values of the first parameter comply with a predetermined first level acceptability criterion, and
if the values of the first parameter comply with the predetermined first-level acceptability criterion, repeating the execution of the first level procedure; and
executing a second level procedure if the values of the first parameter do not comply with the predetermined first level acceptability criterion, the second level procedure comprising:
determining, based at least in part on indications relative to the alteration of the second parameter present in each respective subgroup detected by the second level sensor, which subgroups comply with a predetermined second level acceptability criterion.

49. The computer-implemented method of claim 48, wherein the first and second level sensors are different types of sensors.

50. The computer-implemented method of claim 48, wherein the load volume comprises a plurality of subgroups of products, each subgroup being identifiable and distinct from the other subgroups.

51. The computer-implemented method of claim 48, wherein the method further comprises certifying that all the products in the load volume comply with a predetermined quality standard if, in a reference time interval during which the first level procedure is performed a plurality of times, the values of the first parameter comply with the predetermined first level acceptability criterion during each execution of the first level procedure.

52. The computer-implemented method of claim 48, wherein the method further comprises certifying that the products in the load volume meet a predetermined quality standard if, in a reference time interval during which the first level procedure is performed a plurality of times, one or more of the following conditions are met:
the second level procedure is never performed; or
the second level procedure is performed and a determination is made that all subgroups comply with the predetermined second level acceptability criterion.

53. The computer-implemented method of claim 48, wherein the second level procedure further includes reporting subgroups that do not comply with the predetermined second level acceptability criterion.

54. The computer-implemented method of claim 49, wherein the first parameter is detected at the position inside the load volume in which the first level sensor operates.

55. The computer-implemented method of claim 48, wherein either:
the predetermined first level acceptability criterion is defined by a range of temperatures, or by a range of parameter acceptability values as a function of temperature or temperature variation; or
the predetermined first level acceptability criterion is defined by a concentration range or concentration variation of a prefixed gas or gas mixture.

56. The computer-implemented method of claim 48, wherein the second parameter is at least one of:
a temperature or a temperature-dependent parameter, of each package or of an environment inside each package;
a temperature of an outer surface of the package;
a temperature inside each package;
a concentration or a concentration variation of a predetermined gas inside the package;
a concentration or a concentration variation of a predetermined substance inside the package; or
a degree of acidity present inside the package or a variation of the degree of acidity in each package.

57. The computer-implemented method of claim 48, wherein the second level sensor provides that the predetermined second level acceptability criterion is considered to be complied with for a given subgroup (13) only if the indication provided by the second level sensor used in the second level procedure for that subgroup complies with a respective verification test.
